(19) United States
(12) Patent Application Publication
(10) Pub. No.: US 2024/0114895 A1
(43) Pub. Date: Apr. 11, 2024

(54) IN VIVO GRAFT PERFUSION

(71) Applicant: The Cleveland Clinic Foundation, Cleveland, OH (US)

(72) Inventors: Bahar Bassiri Gharb, Westlake, OH (US); Antonio Rampazzo, Westlake, OH (US); Varun Lingaiah Kopparthy, Morrisville, NC (US)

(21) Appl. No.: 18/376,023

(22) Filed: Oct. 3, 2023

Related U.S. Application Data
(60) Provisional application No. 63/413,291, filed on Oct. 5, 2022.

(51) Int. Cl. A01N 1/02

(52) U.S. Cl. CPC A01N 1/0247; A01N 1/0226

(57) ABSTRACT

A portable, patient-wearable perfusion system includes a patch configured to cover at least a portion of a biological tissue graft implanted within a defect in a patient, a perfusion assembly configured to be worn or carried on the patient, and arterial and venous cannulas. The arterial and venous cannulas are configured to yield a closed fluid circuit for the perfusate to circulate through the graft. The patch includes a first sensor for detecting a first parameter relating to a physiologic state of the biological tissue graft. The perfusion assembly includes a reservoir for a perfusate, a pump, and a controller operatively coupled to the sensor. The controller is configured to operate the pump to control the physiologic state of the tissue graft based on data regarding the first parameter. The portable-patient wearable perfusion system may be used to preserve the implanted graft until the body naturally vascularizes the graft.

### CROSS-REFERENCE TO RELATED APPLICATIONS

This application claims the benefit of U.S. Provisional Patent Application Ser. No. 63/413,291 filed Oct. 5, 2022, whose contents are incorporated by reference.

### TECHNICAL FIELD

The present disclosure relates generally to in vivo biological tissue perfusion and, more specifically, to systems and methods that employ a portable, patient-wearable, in vivo, normothermic perfusion system to preserve biological tissue implanted within a defect at an external surface of the patient's body until the body naturally vascularizes the biological tissue.

### BACKGROUND

Free tissue transfer is a well-established technique allowing for the reconstruction of wounds (or defects) following trauma, tissue extirpation, and congenital malformations by transferring tissues with an established vascular supply to the wound site. The conventional free tissue transfer technique involves creating a defect surgically, either following the removal of a tumor or after a wound is cleaned. An incision is made in an area of a patient or a donor from where a transplant tissue flap will be taken. The top layers of the flap are dissected and freed from the surrounding tissue, such that only the underlying vasculature remains connected to the patient or donor. At least one vein and one artery (constituting the vascular pedicle) that supply blood to the flap are dissected. Before the pedicle is divided, the defect is prepared by identifying a recipient artery and vein at the defect location. After the vascular pedicle has been dissected and the recipient artery and vein in the defect have been identified, the vascular pedicle is divided and the flap is completely separated and removed from the patient or donor. When separated and removed, the flap is called a “free flap.” The free flap is brought to the defect area, and the vein and artery from the flap (vascular pedicle) are connected to the recipient vein and artery in the defect by suturing, etc. The process of establishing an anastomosis (i.e., a connection) between the respective vasculature in the flap and the defect can be performed only by highly skilled microsurgeons. The process is both time- and labor intensive and requires the use of expensive equipment, such as an operating microscope called a loupe, given the delicate nature and small size (0.3-3 mm) of the vessels involved. After an anastomosis has been established between respective veins and arteries in the defect and the free flap, the perimeter of the free flap is sutured to the defect and the blood vessels are monitored to ensure that an adequate blood flow between the defect and the free flap is maintained. The area from which the free flap was harvested in the patient or the donor is then closed, often with a Split Thickness Skin graft (STSG).

The most critical step of the conventional free tissue transfer technique is establishing an anastomosis between the vessels of free flap pedicle and the vessels of the defect. The failure rate of free-flap transplants transplanted by existing free tissue transfer procedures, commonly attributed to blood clots, lies around 3-5%. Clotting is ascribed to several factors including procedure technique, poor quality vessels (i.e., radiated or atherosclerotic vessels), poor flow in the vessels, long operative times, hypercoagulative states, etc. If the free flap fails, then the patient is at risk for additional complications, such as infection, delayed wound healing, need for second (or subsequent) surgical procedures, or even amputation in the case of traumatic limb defects with exposed bone. Current research has focused on improving anastomosis procedures and re-repairing vessels after the removal of a clot. In the case of clot removal, new recipient vessels are often used. If that is not possible, the failure rate increases. Rates of successful flap salvage after a clot have been described to be only between 30 to 60%.

Accordingly, it would be desirable to facilitate free-flap transplantation in a manner that avoids the need for time-consuming, highly skilled microsurgery to establish anastomoses, and then the necessary monitoring and follow-up to ensure continued patency of the anastomoses.

### SUMMARY

Disclosed herein is a normothermic machine perfusion (NMP) system that can be utilized in vivo to promote neovascularization of transplanted free flaps within a tissue defect. As will be further described, NMP can maintain the physiologic metabolism of a free flap during neovascularlization, avoiding the deleterious effects of hypoxia (low oxygen levels in the tissues), hypothermia (cooling), and nutrient deprivation that occur with transplant rejection. NMP also can sustain and/or preserve implanted biological tissue within a defect at an external surface of a patient's body without surgical anastomosis between respective artery(ies) and vein(s) in the tissue and the defect, for example at least for four weeks (one month) or until the body naturally vascularizes (neovascularizes) the implanted tissue.

Because NMP preserves and sustains the free flap until the body can neovascularize the free flap, preparation of and anastomosis to defect-(recipient)-site vessels are not required for a successful flap implantation. Moreover, operating times may be substantially reduced with NMP because surgical procedures are not required to establish anastomoses between the free flap and defect vasculature.

NMP may also be used for reconstruction of defects in sick patients who would not or would be less likely to endure long operative procedures, patients with hypercoagulability, as well as patients with scarred and irradiated fields or other risk factors for flap failure. NMP may also be used to salvage flaps that have failed using the conventional techniques. Finally, the use of NMP would circumvent the use of vein grafts necessary to bridge adequate vessels if these are not available in close proximity to the defect, and would present less risk of morbidity to patients. Accordingly, the disclosed normothermic machine perfusion system includes a portable, patient-wearable perfusion system that can perfuse an implanted biological tissue graft (e.g., a free flap) to preserve the implanted biological tissue graft without surgically establishing anastomoses between respective vasculature in the graft and the defect. Methods of using such a system also are disclosed.

A portable, patient wearable, perfusion system can extend the life of a biological tissue graft implanted within a defect at an external surface of a patient's body, ensuring that the implanted biological tissue graft is preserved until the body neovascularizes the reattached graft.

In an aspect, the present disclosure includes a portable, patient-wearable perfusion system that includes a patch configured to cover at least a portion of a biological tissue graft implanted within a defect in a patient, a human-portable perfusion assembly configured to be worn or carried on the patient's body, an arterial cannula, and a venous cannula. The patch includes a first sensor adapted to detect a first parameter relating to a physiologic state of the biological tissue graft. The perfusion assembly includes at least one reservoir for a perfusate, a pump, and a controller operatively coupled to the sensor and configured to operate the pump to control said physiologic state of the tissue graft based on data regarding the first parameter received from the sensor. The arterial cannula is configured to cannulate a major artery of the biological tissue graft to facilitate delivery of perfusate to the graft from the perfusion assembly. The venous cannula is configured to cannulate a major vein of the biological tissue graft to facilitate return of spent perfusate from the graft back to the perfusion assembly. The arterial cannula and the venous cannula are configured to yield a closed fluid circuit for the perfusate to circulate through the biological tissue graft.

In another aspect, the present disclosure includes a method for preserving an implanted biological tissue graft. The method includes connecting an external perfusion assembly located outside of a patient's body to a major artery and a major vein of a biological tissue graft via respective arterial and venous cannulas, thereby establishing a closed fluid circuit for a flow of perfusate between the tissue graft and the perfusion assembly, and perfusing the implanted biological tissue graft in vivo via perfusate circulating through the closed fluid circuit.

### BRIEF DESCRIPTION OF THE DRAWINGS

The foregoing and other features of the present disclosure will become apparent to those skilled in the art to which the present disclosure relates upon reading the description with reference to the accompanying drawings, in which:

FIG. 1 is a schematic view of a portable, patient-wearable perfusion system.

FIG. 2 is a more detailed schematic view of an example embodiment of a portable, patient-wearable perfusion system.

FIGS. 3-5 are schematic views of portions of the example of FIG. 3.

FIG. 6 shows an example portable, patient-wearable perfusion system connected to an implanted biological tissue graft.

FIG. 7 is a process flow diagram of an example implementation of the system of FIG. 1.

FIG. 8 is a process flow diagram of an exemplary method for exchanging perfusate.

FIG. 9A is a view of one embodiment of an arterial cannula.

FIG. 9B is a view of one embodiment of a venous cannula.

FIG. 10 is a cross-sectional view of an embodiment of a negative pressure venous return system.

FIG. 11 is a schematic view of an example patch containing arrays of sensors.

### DETAILED DESCRIPTION

### I. Definitions

In the context of the present disclosure, the singular forms “a,” “an” and “the” can also include the plural forms, unless the context clearly indicates otherwise.

The terms “comprises” and/or “comprising,” as used herein, can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “implanted biological tissue graft” refers to any body part, or vascular allograft or autograft originating from a donor or a patient or grown in a laboratory setting that has been implanted, such as by suturing, stapling, etc., within a defect formed (e.g., due to trauma, disease, etc.) at an external surface of the body of the patient.

As used herein, the term “detached” can refer to the state of something that was once attached no longer being attached. For example, skin tissue can be detached from a patient or a donor due to trauma or as part of a surgical procedure.

As used herein, the term “implanted” can refer to the state of something that was once detached now being attached. For example, detached skin tissue may be implanted within a defect in a patient that was formed by trauma or as part of a surgical procedure by suturing the free flap around the perimeter of the defect. As used herein, the term “implanted” does not necessarily include establishing an anastomosis between the artery(ies) and/or vein(s) of any biological tissue, extremity, allograft or autograft to any respective artery(ies) and/or vein(s) of the defect.

As used herein, the term “ex-vivo” (when used to refer to a body part) refers to the body part being outside or separated from the patient or donor, as opposed to being inside or implanted in the patient or donor under normal living conditions. In contrast, as used herein the term “in-vivo” (when used to refer to a body part) refers to the body part being inside or implanted within the patient or donor under normal living conditions. The ex-vivo or in-vivo body part can include, but is not limited to, organs such as the heart, kidney, liver, lungs, pancreas, intestines, or skin.

As used herein the terms “free flap,” “free autologous tissue transfer,” and “microvascular free tissue transfer” refer to a piece of tissue that is disconnected from its original blood supply and is moved a distance to be implanted at a recipient site (i.e., a defect). The free flap can originate with a patient, a donor, or a culture in a laboratory setting.

As used herein, the term “patient” refers to any warm-blooded organism (e.g., a human being, a primate, a cat, a dog, a rabbit, a mouse, etc.) receiving treatment for a medical condition that requires a transplant or replant of a detached biological tissue. For example, a patient may require a skin transplant or replant due to an injury or a disease. A patient can be in any location, a hospital, a doctor office, a field hospital, etc.

As used herein, the term “donor” refers to any warm-blooded organism, living or dead, that undergoes a surgical procedure to detach a biological tissue that will be transplanted to a patient. The patient may be the same or different from the donor.

As used herein, the term “normothermic” refers to an environmental temperature that does not cause increased or decreased activity of cells of a body. For a human body the peak normothermic temperature range is between approximately 36 degrees Celsius and 38 degrees Celsius.

As used herein, the term “perfusate” refers to a fluid comprising nutrients, substrates, metabolites, electrolytes, and/or an oxygen carrier that is perfused through implanted biological tissue graft to preserve the function and viability of the implanted biological tissue graft.

As used herein, the term “substrate” refers to one or more materials that are added to a perfusate to help nourish the cells in implanted biological tissue.

### II. Systems

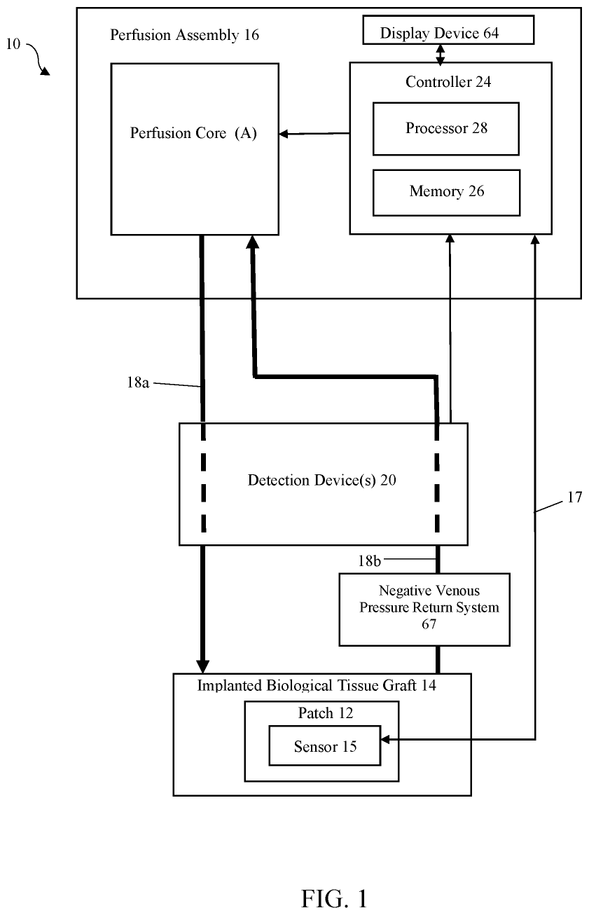

An aspect of the present disclosure can include a portable, patient-wearable system 10 as shown in FIG. 1. The system 10 employs a perfusion assembly 16 that can be worn or carried on the patient's body and used, for example, to preserve an implanted biological tissue graft 14 until the body can neovascularize the graft. The implanted biological tissue graft 14 may be a vascularized composite autograft (e.g., free flaps or free tissue transfer). When the implanted biological tissue graft 14 is a vascularized composite allograft, the vascularized composite allograft may be a free flap. The system 10 can provide a normothermic environment for the implanted biological tissue graft 14 that mimics the physiological environment of the biological tissue graft 14 to preserve the graft and to prevent ischemia, hypoxia, or other deleterious outcomes from affecting the graft during neovascularization. The compactness and mobility of the system 10 combined with longer preservation times from normothermic machine perfusion can maintain the viability of an implanted biological tissue graft 14 without requiring surgical anastomosis until the body can neovascularize the tissue.

The portable and patient wearable perfusion system 10 includes a patch 12 having a sensor 15 that is sized and dimensioned to cover at least a portion of the external surface of the implanted biological tissue graft 14. The system also includes a perfusion assembly 16 adapted to maintain the normothermic environment for the implanted biological tissue graft 14 by pumping a perfusate through the graft. A normothermic environment mimics at least one of the physiologic temperature, pressure, and humidity of the implanted biological tissue graft 14 to decrease the onset of cellular damage and to elongate tissue survival time. An arterial tube 18a can be adapted to connect the perfusion assembly 16 to a major artery of the implanted biological tissue graft 14 via an arterial cannula. A venous tube 18b can be adapted to connect the perfusion assembly 16 to a major vein of the implanted biological tissue graft 14 via a venous cannula, optionally after passing the used perfusate through a negative pressure venous return system 67.

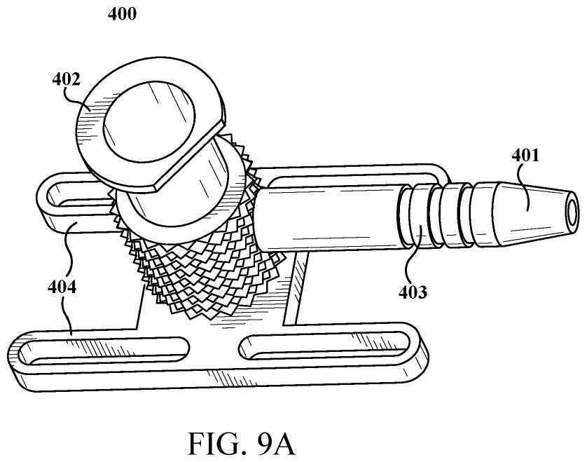

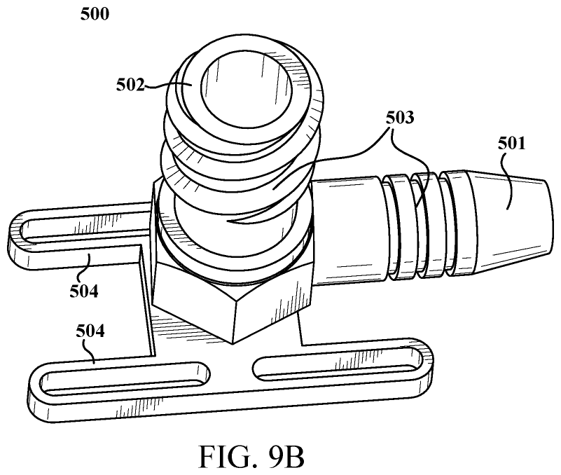

Exemplary arterial 400 and venous 500 cannulas are illustrated in FIGS. 9A and 9B, respectively. The cannulas possess generally tubular portions for cannulating the respective blood vessels, having insertion ends 401, 501 configured for cannulation (i.e., introduction) into an interior lumen of a respective major artery or major vein. Ports 402, 502 of the respective cannulas are configured to be connected to the respective arterial or venous tube. The port 502 of the venous cannula 500 may also be configured to connect to an inlet of a negative pressure venous return system, discussed below. The insertion ends 401, 501 and ports 402, 502 are in fluid communication with one another, respectively, so that perfusate, blood or other liquid may flow between them in the respective cannula 400, 500.

The insertion ends 401, 501 and/or ports 402, 502 may contain one or more attachment features 403, 503 on the surface thereof to detachably secure or connect the cannulas to the interior lumen of the respective vessel or tube or inlet by friction or press-fit. For example, the attachment features 403 may include one or more of lipped edges, screw threads, indentations, protrusions, or grooves. In an embodiment of an arterial cannula shown in FIG. 9A, the arterial cannula 400 has attachment features 403 in the form of a series of indentations or grooves on the insertion end 401. In an embodiment of a venous cannula shown in FIG. 9B, the venous cannula 500 has attachment features 403 in the form of a series of indentations or grooves on the insertion end 501 and a screw thread associated with the port 502. The connection between the insertion ends 401, 501 and the respective vessel may be externally secured using a suture tie or a clip mechanism to grip the vessel to the cannula.

The cannulas 400, 500 may be formed in various sizes and lengths to accommodate the particular application, such as the size of the vessel(s) to be cannulated. The cannulas 400, 500 may be made from various medical-grade materials, including stainless steel, titanium, or plastics, such as polytethylene or polypropylene.

In the embodiments illustrated in FIGS. 9A and 9B, the ports 402, 502 of the cannulas are arranged perpendicular (i.e., at a substantially 90-degree angle) to the tubular portions having the respective insertion ends 401, 501, thereby a forming an L-shaped cannula with an interior lumen capable of allowing perfusate, blood or other liquid to flow therethrough. When the insertion ends 401, 501 are connected to their respective vessels and a biological tissue graft is implanted on a flat, horizontal defect on the patient, medical professionals can easily access the ports 402, 502 of the L-shaped cannulas to attach or detach the respective arterial or venous tubing. However, the arrangement of the insertion ends 401, 501 and ports 402, 502 is not so-limited, and they may be arranged at a desirable angle to facilitate access to the ports 402, 502 and/or flow of perfusate depending on the location of the defect in the patient's body (i.e., arm, leg, trunk). For example, the insertion ends 401, 501 and the ports 402, 502 may be arranged to provide a cannula that extends in a straight line (i.e., a substantially 180-degree angle) or to provide a cannula that has a 120-degree angle between the insertion end and port.

In the illustrated embodiments, the cannulas 400, 500 are arranged respectively on a pair of feet 404, 504 configured to be co-planar with the surface of the biological tissue graft or the patient's skin. The feet 404, 504 are further configured to rest against the surface of the biological tissue graft and to stabilize the cannula in relation thereto. The shape and dimensions of the feet 404, 504 are not limited and may be selected or configured to promote stabilization based on the region of the patient's body where the defect is located. The feet may be secured to the biological tissue graft or the patient's skin surrounding the defect with, for example, surgical tape or sutures. In the embodiments shown in FIGS. 9A and 9B, feet 404, 504 contain a pair of linear openings through which sutures may be stitched to affix the cannulas against the biological tissue graft or the patient's skin.

Returning to FIG. 1, the arterial cannula and the venous cannula, when connected to arterial tube 18a and the venous tube 18b and cannulated to the major artery and major vein of the implanted biological tissue graft 14, are configured to yield a closed-loop fluid circuit for the perfusate to circulate through the biological tissue graft 14. In this manner, perfusate is circulated through the native vasculature of the implanted biological tissue graft 14 via its cannulated arterial and venous vessels in a closed loop, in order to supply nutrients, oxygenation, and optionally other medicinal or metabolic agents that will help sustain the graft until the patient's body neovascularizes it within the defect where it was implanted. This system and methodology foregoes the need to establish anastomoses between the graft and the patient. Instead, vascularization of the graft to the patient will happen pursuant to the patient's natural neovascularization processes, while the graft is sustained and supported via perfusate supplied via the perfusion assembly 16. The closed-loop fluid circuit described here differs from other ex vivo perfusion systems, where the vein of the biological tissue graft is not cannulated such that used perfusate free-flows out from the venous system of the graft in an unconfined manner; e.g. via gravity into a drain, a cuff or other collection receptacle.

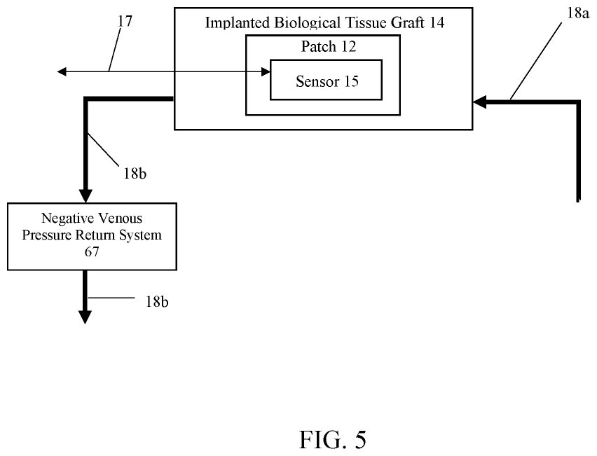

The arterial and venous tubes 18a, 18b are indicated in FIGS. 1-2 and 4-6 using bolded lines (dashed where the tubing can pass through another component of the system) with arrows indicating the direction of perfusate flow in the tubes. The arterial and venous tubes 18a, 18b can be, for example, polyvinyl tubing, silicon tubing, or any other tube-like structure with an interior lumen that can allow the flow therethrough of a liquid or gas.

The patch 12 may have any dimensions suitable to cover at least a portion of an external surface of the implanted biological tissue graft 14. The patch, for example, may be about 30 cm by about 15 cm, about 25 cm by about 10 cm, or about 20 cm by about 8 cm. In a one embodiment, the patch 12 may be configured to cover the entire implanted biological tissue graft 14. In another embodiment, the patch 12 can be configured to cover a specific type of biological tissue graft 14, for example a free flap implanted within a defect formed in a patient's chest after a mastectomy. Alternatively, the patch can be configured as a universal covering for any type of implanted biological tissue graft 14.

The patch 12 may be made of any suitable materials conventionally known in the art. For example, the patch 12 may be composed of a non-woven material, such as rayon or polyester, a woven material, a plastic or vinyl film, a silicone, a foam, an alginate, a gel, a hydrogel, or a combination thereof. In one embodiment, the patch 12 has a suitable medical-grade adhesive disposed on the bottom (skin-facing) perimeter surface to adhere the patch to the implanted biological tissue graft 14. Alternatively, the patch 12 does not include an adhesive and may be secured to the external surface of the implanted biological tissue graft 14 or another portion of the patient's body by applying a suitable medical-grade tape about the perimeter of the top surface of the patch 12.

At least one sensor 15 is included in the patch 12. When the patch 12 is applied, the sensor 15 directly contacts an external surface of the implanted biological tissue graft 14. In one embodiment, the at least one sensor 15 can be integrally formed or incorporated within the patch 12, such that the patch 12 and the at least one sensor 15 can form a single unit. In another embodiment, the at least one sensor 15 is provided separately from the patch 12 but is attached thereto (e.g., by an adhesive). In this embodiment, the at least one sensor 15 may be applied onto the bottom (skin-facing) surface of the patch 12, and then the patch 12 and sensor 15 may be applied to the implanted biological tissue graft 14, such that the sensor 15 and the bottom surface of the patch 12 contact at least a portion of the external surface of the graft.

The at least one sensor 15 can be at least one of: a tissue oximeter, a tissue temperature sensor, and a tissue optical color sensor. These or other sensors are adapted to measure characteristics of the implanted biological tissue graft 14, which then can be analyzed by a controller 24 of the perfusion assembly 16 to provide feedback control in order to adjust perfusion, control the delivery of composition of perfusate, or even to determine when it is safe to cease perfusion using perfusate. For example, a tissue oximeter can be adapted to measure oxygen saturation in one or more regions of the implanted biological tissue graft 14. In this example, when the implanted biological tissue graft 14 is a flap of skin tissue (for instance), the tissue oximeter can be a near infrared sensor on the surface of the skin tissue that is held in-place by the patch to detect tissue oxygenation at different depths within the flap of skin tissue. As will be discussed in further detail below, a degree of neovascularization of the implanted biological tissue graft 14 may be determined by stopping the flow of perfusate to the graft and measuring tissue oxygenation via one or more tissue oximetry sensors. A value of at least 70%, at least 80%, at least 90% or at least 99% tissue oxygen saturation suggests that the graft is being sufficiently oxygenated. Sufficient oxygenation indicates that the implanted biological tissue graft 14 is being neovascularized under existing conditions. A value of less than 40% tissue oxygen saturation indicates that the implanted biological tissue graft 14 is insufficiently oxygenated and that neovascularization is likely not occurring.

The tissue temperature sensor can be adapted to measure the surface temperature in one or more regions of the implanted biological tissue graft 14. For example, the tissue temperature sensor can be a thermistor, a semi-conductor sensor, or an infrared temperature sensor. In embodiments, temperature values in a range of between about 33° C. and about 38° C. indicate normothermia suggesting that the implanted biological tissue graft 14 is being neovascularized under existing conditions. A temperature value lower than 33° C. will indicate that the biological tissue graft 14 is insufficiently vascularized and that neovascularization is not likely occurring.

The tissue optical color sensor can be adapted to detect one or more colors of the implanted biological tissue graft 14. For example, the optical color sensor can emit light from a transmitter onto the implanted biological tissue graft 14, and then detect the light reflected back from the graft with a receiver in order to determine the color(s) of the graft. Detected color may be correlated to a degree of neovascularization of the implanted biological tissue graft 14, gain after temporarily discontinuing perfusate flow so that graft temperature is affected based on the degree of vascularized blood flow, and not the degree of perfusate flow. In one embodiment, the detection of warmer colors (i.e., reds, pinks and oranges) by the color sensor indicates that the biological tissue graft 14 is adequately oxygenated and likely neovascularizing under existing conditions, while the detection of cooler colors (i.e., greens, blues or purples) indicate that the biological tissue graft is inadequately oxygenated and likely not undergoing neovascularization.

In one embodiment, the patch 12 may include one or more sensor arrays. Each sensor array includes a plurality of the same type of sensor 15 (e.g., a tissue oximeter, a tissue temperature sensor, a tissue optical color sensor) and is adapted to detect respective values of the same parameter from a multitude of corresponding locations in the implanted biological tissue graft 14. The data regarding the parameter generated by each sensor 15 in the array is received by a controller 24, which is configured to process the respective values regarding that parameter in order to control the physiologic state of the implanted biological tissue graft 14 based thereon. In one example, the patch 12 includes an array of temperature sensors adapted to detect respective temperature values across the external surface of the implanted tissue graft 14.

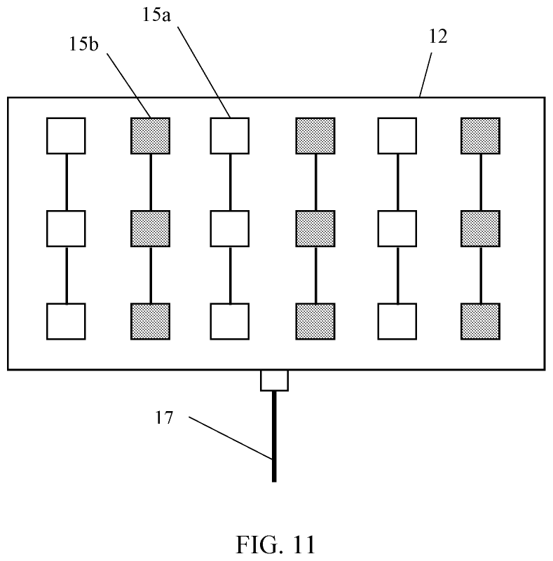

When two arrays of different sensors are used, the controller 24 is configured to process the respective values of the second parameter detected by the second array of sensors together with those of the first parameter. In one embodiment shown in FIG. 11, the patch 12 includes a first array 15a of sensors 15 that includes temperature sensors adapted to detect temperature, and a second array 15b of sensors 15 that includes tissue oximeters adapted to detect oxygen saturation. As shown in FIG. 11, the first and second arrays of sensors 15a, 15b can be distributed in alternating rows of the respective sensors to distribute them substantially uniformly across the patch 12.

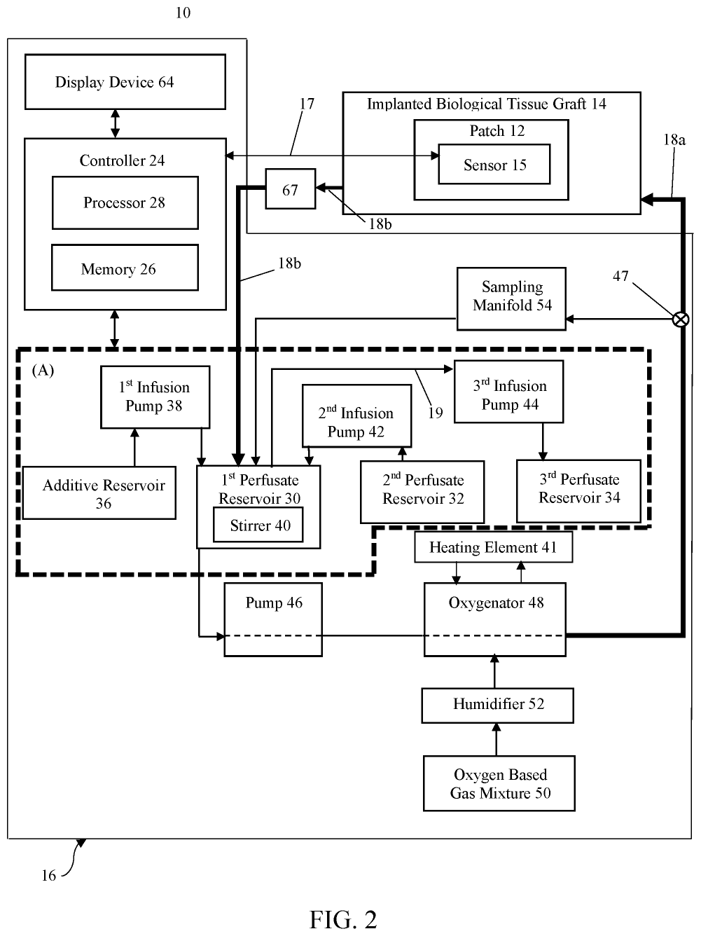

As used herein, the term “perfusion assembly” refers to a mechanical system for perfusing a perfusate through biological tissue that has been implanted within a defect (e.g. at an external surface) of a patient's body. Referring to FIGS. 1-3, perfusion assembly 16 can include a perfusion core (A), one or more parameter control devices (e.g., pump 46, heating element 41, oxygenator 48), a display device 64, and a controller 24 connected to the display device 64 and/or to the at least one sensor 15 of the patch. As used herein, the term “perfusion core” (A) refers to the portion of the perfusion assembly 16 that includes one or more perfusate reservoirs 30, 32, 34 adapted to store and adjust characteristics of the perfusate that is circulated through the implanted biological tissue graft 14, as well as one or more additive reservoirs 36, conduits 19 for transporting perfusate through the perfusion core (A), and one or more parameter control devices (such as infusion pumps 38, 42, 44 for pumping additives and perfusate between reservoirs). Other parameter control devices (such as heating element 41, oxygenator 48. humidifier 52 and/or oxygen-based gas mixture 50) also may be included within the perfusion core (A), e.g. as part of an integrated assembly therewith. These and other parameter control devices also can be supplied separately (as shown in FIG. 2), not as part of the perfusion core (A). Conduits 19 are indicated in FIGS. 2-4 with normal (i.e., non-bolded) lines with arrows indicating the direction of perfusate flow throughout the perfusion assembly 16, and dashed where the conduits can pass through another component of the system. The perfusion core (A) of the perfusion assembly 16 is indicated with a bolded and dashed lined box (A).

The perfusate that can be perfused through the system 10 and the implanted biological tissue graft 14 can be any suitable perfusate. For example, the perfusate can include a colloid solution with physiologic concentrations of albumin, glucose, electrolytes, and an oxygen carrier (e.g., washed red blood cells). In another example, the perfusate is specifically formulated for the preservation of vascular composite allografts.

The perfusion assembly 16 can also include at least one detection device 20. The at least one detection device 20 can measure a parameter during perfusion of the implanted biological tissue 14. Unlike the at least one sensor 15, which is included in the patch 12, the at least one detection device 20 is instead included within, or attached to, the perfusion assembly 16 or to features of the system 10 physically outside of the perfusion assembly 16 but operatively connected thereto, other than patch 12 (i.e., the arterial tube 18a and/or the venous tube 18b as shown in FIG. 1), such that the detection device(s) 20 will detect parameters of the perfusate used to perfuse the graft 14 in order to infer characteristics of the graft while undergoing neovascularization, as opposed to the sensor 15 in the patch 12, which directly detects features of the graft itself. Each detection device 20 can be positioned in the same or different portions of the system 10, excluding the patch 12 or the implanted biological tissue graft 14, depending on the parameter to be detected. For example, one such detection device 20 may be positioned in the perfusion assembly 16, while another detection device 20 may be positioned on or within the arterial tube 18a and/or the venous tube 18b. As shown in FIG. 1, a one or more detection device(s) 20 may be connected to, or configured to detect parameters associated with perfusate flowing through, both the arterial tube 18a and the venous tube 18b. The parameter measured by the at least one detection device 20 can be a metabolic parameter or a physiologic parameter depending on the type and location of the at least one detection device 20. Metabolic parameters can include, but are not limited to, glucose concentrations and lactate concentrations measured in the perfusate housed in the perfusion assembly 16 or the arterial and/or venous tubes 18a, 18b. Physiologic parameters can include, but are not limited to, temperature of the implanted biological tissue graft 14 (inferred from the temperature measured in the perfusate at a location remote from the graft); pressures within the arterial and venous tubes 18a, 18b; and oxygen saturation in the implanted biological tissue graft 14 (again inferred from that in the perfusate at the location where measured). The at least one detection device 20 can be, but is not limited to, one or more of a flow-through biosensor, an in-line biosensor, an electrochemical sensor, an electro-optical sensor, or a pH sensor.

The at least one detection device 20 can be at least one of: a tissue oximeter, a temperature sensor, a pressure sensor, a pH sensor, an ion-selective electrode, a flow-sensing module, and a sensor adapted to measure at least one of metabolite concentrations and/or blood gas concentrations in the perfusate. When the at least one detection device 20 is a temperature sensor, the temperature sensor can be adapted to measure the temperature of the perfusate at a location anywhere in the system 10. For example, the temperature sensor can be a thermistor, a semi-conductor sensor, or an infrared temperature sensor. In a preferred embodiment, the temperature of the perfusate will be in the range of between about 33° C. and about 38° C.

A pressure sensor can be adapted to measure the pressure of the perfusate when the perfusate is at a location anywhere in the system 10. For example, a pressure sensor placed on the arterial tube 18a can be configured to measure the in-line arterial pressure. In a preferred embodiment, the in-line arterial pressure should measure between about 30 mmHg to about 90 mmHg, about 35 mmHg to about 85 mmHG, or about 40 mmgHg to about 80 mmHg.

A pH sensor can be adapted to continuously, or at times specified by a user of the system 10 (e.g., a medical caregiver or patient), measure the pH of the perfusate in at least one of the perfusion assembly 16, the arterial tube 18a, and the venous tube 18b. In a preferred embodiment, the pH of the perfusate is within a range of about pH 7 to about pH 8, about pH 7.15 to about pH 7.75, or about pH 7.30 to about pH 7.50.

An ion-selective electrode can be adapted to measure electrolyte concentrations (e.g., Na+, K+, Ca++, etc.) in the perfusate in the perfusion assembly 16, the arterial tube 18a and/or the venous tube 18b. In an embodiment, the concentration of Na+ is between 125 mmol/L to about 170 mmol/L, about 130 mmol/L to about 165 mmol/L, or about 135 mmol/L to about 160 mmol/L. In another embodiment, the concentration of K+ in the perfusate is between about 1 mmoL/L to about 30 mmol/L, about 1.5 mmol/L to about 25 mmol/L, or about 2 mmol/L to 20 mmol/L. In another embodiment, the concentration of Ca++ in the perfusate is between about 0.25 mmol/L to about 10 mmol/L, about 0.5 mmol/L to about 7.5 mmol/L, or about 1 mmol/L to about 5 mmol/L.

A flow sensing module can be adapted to continuously, or at times specified by a user of the system 10, measure the inflow and outflow of perfusate non-invasively. The flow sensing module may be an ultrasound sensor configured to detect real-time flow rate.

A sensor adapted to measure metabolite concentrations and/or blood gas concentrations in the perfusate can be positioned in at least one of the perfusion assembly 16, the arterial tube 18a, and the venous tube 18b. The measured metabolite concentrations can include, but are not limited to, glucose concentrations and lactate concentrations. Not wishing to be bound by theory, glucose concentrations can be used to assess the metabolic function of the implanted biological tissue graft 14 by determining the amount of glucose used by the implanted biological tissue graft 14 to generate energy. Lactate is a marker of anaerobic activity and, not wishing to be bound by theory, lactate concentrations can be used to determine the occurrence of injuries. In an embodiment, the glucose concentration is between about 10 g/dL to about 300 g/dL, about 20 g/dL to about 250 g/dL, or about 30 g/dL to about 200 g/dL. In another embodiment, the lactate concentration is between about 0.5 mmol/L to about 45 mmol/L, about 1 mmol/L to about 30 mmol/L, or about 2 mmol/L to about 15 mmol/L.

The measured blood gas concentrations can include, but are not limited to, oxygen and carbon dioxide concentrations. For example, the oxygen concentration (pO₂) is between about 50 mmHg to about 900 mmHg, about 75 mmHg to about 850 mmHg, or about 100 mmHg to about 800 mmHg. In another example, the carbon dioxide concentration (pCO₂) is between about 10 mmHg to about 200 mmHg, about 15 mmHg to about 150 mmHg, or about 20 mmHg to about 100 mmHg. Other metabolite and blood gas concentrations that indicate the health of an implanted biological tissue graft 14 can also be measured. More than one type of each detection device 20 can be positioned in the system 10 when the at least one detection device 20 is more than one.

The at least one parameter control device can maintain or adjust a parameter to fall within a predetermined threshold. A parameter control device can be at least one of: a pump, an oxygenator, a heating element, and a source of gas mixture. In the embodiment illustrated in FIG. 2, there are parameter control device(s) (in the form of infusion pumps 38, 42 and 44) encompassed within the perfusion core (A), and others (such as the pump 46, oxygenator 48, heating element 41, humidifier 52 and oxygen based gas mixture 50) located outside the perfusion core (A) but still part of the perfusion assembly 16. Additional parameter control device(s) may also be physically located outside of the perfusion assembly 16 but operatively connected and/or attached thereto, such as a heating element attached to arterial tubing 18a. In the embodiment shown in FIG. 2, the pump 46 can be adapted to control the overall mass flow rate of perfusate that will circulate into the tissue graft 14 via the arterial line 18a (via the oxygenator 48 as-illustrated). Other parameter control devices (e.g. infusion pumps 38, 42 and 44) may be used separately to control metabolite concentrations in the perfusate, by regulating the rate at which spent perfusate is removed from the first perfusate reservoir 30 or the rate at which solutions in other reservoirs are fed therefrom to the first perfusate reservoir 30 from which pump 46 draws perfusate to circulate into the graft 14 via arterial line 18a. Such solutions may include additives such as a substrate, nutrient, electrolyte, or buffer, which can be added into the first perfusate reservoir 30, which stores the perfusate that is to be circulated into the graft 14 via pump 46. The pump 46 can be, but is not limited to, at least one of an infusion pump, a peristaltic pump, or a roller pump. The oxygenator 48 can be adapted to oxygenate the perfusate as the perfusate flows from the perfusion core 16 through the at least one conduit 19 towards the implanted biological tissue graft 14. The oxygenator 48 may be removable and replaceable.

A heating element 41 is another example of a parameter control device that can be used to regulate perfusate temperature, e.g. as it flows through the oxygenator 48 in the embodiment of FIG. 2. Such a heating element 41 can be adapted to help maintain the implanted biological tissue graft 14 at a normothermic temperature by regulating the temperature of the perfusate that circulates into the graft 14 via the arterial tubing 18a. For example, the heating element 41 may be connected to the oxygenator 48 or to the arterial tube 18a. When connected to the arterial tube 18a, the heating element 41 may be positioned at a location along the length of the tube or adjacent to the port 402 of the arterial cannula 400 to ensure that the temperature of the perfusate is within the desired normothermic range just before it enters the biological tissue graft 14. For example, the heating element 41 may be programmed by the controller 24 to heat the perfusate in the arterial tubing to a temperature of 35° C. The heating element 41 may contain a thermoelectric module or one or more cartridge heaters to precisely regulate the temperature of the perfusate. When the at least one detection device 20 includes a temperature sensor, the temperature sensor and the heating element 41 may operate in a feedback loop via the controller 24 to measure and control and temperature of the perfusate flow throughout the system 10.

A source of a gas mixture 50 can be connected to the oxygenator 48 and adapted to provide a quantity of the gas mixture to the perfusate and to maintain a desired pH level of the perfusate. The gas mixture can be a combination of at least oxygen and carbon dioxide (e.g., in some instances, oxygen is much more prevalent than carbon dioxide in a hyperoxygenated environment; for example, O₂ can be more than 50%, more than 75%, more than 85%, and more than 95%; one non-limiting example is 97.5% O₂ and 2.5% CO₂).

As shown in FIGS. 1-3, the controller 24 may be included within the perfusion assembly 16, or it may be positioned outside of the perfusion assembly 16 but operatively connected and/or attached thereto. Controller 24 has a non-transitory memory 26 storing executable instructions (i.e., algorithms) and a processor 28 to execute the instructions stored in the memory. The controller 24 can be coupled to the at least one sensor 15, the perfusion core (A), the arterial and/or venous tubes 18a, 18b, the at least one detection device 20, and/or at least one parameter control device. The controller 24 may also be wirelessly coupled with a cloud-based server to wirelessly transmit data (i.e., data regarding parameters sensed by the sensors 15 and/or the least one detection device 20, detected analyte concentrations, rate of perfusion, etc.) to the cloud-based server in order to permit a medical caregiver or the patient to remotely access the data and monitor the system 10.

In some instances, one or more of the couplings can be via a wired connection. For example, as illustrated in FIGS. 1, 2 and 5, the at least one sensor 15 is coupled to the controller 24 via wired connection 17. In other instances, one or more of the couplings can be via a wireless connection. In still other instances, one or more of the couplings can be via a connection that is both wired and wireless. Similarly, in some instances, the one or more couplings can be via a wireless connection and/or a wired connection. Additionally, each element of the system 10 may have additional components to aid in the coupling that are not illustrated.

The non-transitory memory 26 of the controller 24 can store machine executable instructions (algorithms), which are executable by the processor 28. In some instances, the non-transitory memory 26 can be combined in a single hardware element (e.g., a microprocessor), but in other instances, the non-transitory memory and the processor can include at least partially distinct hardware elements. The controller 24 can be configured to receive data regarding the parameters measured by the at least one sensor 15 and, optionally, the detection devices 20, and may be configured to compare the data to predetermined thresholds or threshold ranges for that parameter. The controller 24 may also be adapted to direct the parameter control devices to control the physiological state of the tissue graft, such as by maintaining or adjusting a property of the perfusate (i.e., a concentration of an additive in the perfusate supplied from a separate additive reservoir of the external perfusion assembly), or the rate of perfusion. Based on the parameter(s) received from the at least one sensor 15 and/or the at least one detection device 20, the controller may also send an alert to be displayed on a display device 64.

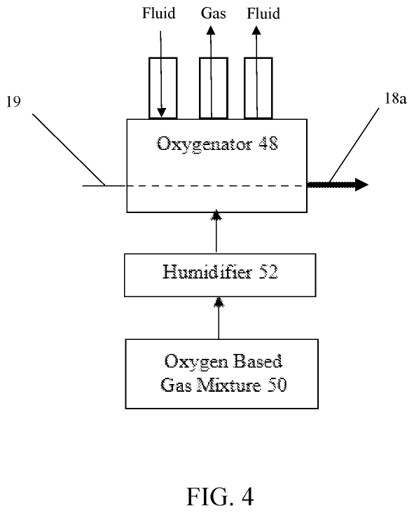

FIG. 2 shows a detailed example of an embodiment of the system 10 and the path the perfusate can take when pumped from the perfusion assembly 16 through the arterial and arterial tube 18a to the implanted biological tissue graft 14, and then back to the perfusion assembly 16 via venous tube 18b. In this embodiment, the components included in the perfusion assembly 16 are outlined with a normal (i.e., not bolded or dashed) lined box. The perfusion core (A) of the perfusion assembly 16, shown in detail in bolded and dashed lined box (A) and in FIG. 4, can include a first perfusate reservoir 30, a second perfusate reservoir 32, and a third perfusate reservoir 34 connected by one or more conduits 19. The controller 24 can be adapted to facilitate the exchange of perfusate between the first perfusate reservoir 30, the second perfusate reservoir 32, and the third perfusate reservoir 34 using at least one parameter control device (e.g., at least one infusion pump 38, 42, 44).

In the example shown in FIG. 2, the first perfusate reservoir 30 provides the source of perfusate that is circulated into the implanted biological tissue graft 14 via pump 46 through arterial tube 18a, as well as the sump of spent perfusate that is returned to the system 10 from the graft 14 via the venous tube 18b. The first perfusate reservoir 30 can also be connected to at least one additive reservoir 36 via at least one conduit 19 and a first infusion pump 38, where at least one additive, such as a substrate, nutrient, electrolyte, or other compound, can be added to the perfusate in the first perfusate reservoir and mixed with the perfusate via a stirrer 40, such as a magnetic stirrer, inside or adjacent to the first perfusate reservoir. Other additive compounds can include sodium bi-carbonate NaHCO₃. The additives are added on-demand to adjust for the desired parameters for the perfusate and to maintain ideal physiological conditions. The additives also aid in maintaining the ideal ranges of electrolytes and metabolites described above (e.g., Na+, K+, Ca++, glucose, lactate, etc.). The second reservoir 32 as shown can be adapted to hold cooled perfusate and can be in fluid communication with the first perfusate reservoir 30 via at least one conduit 19 and a second infusion pump 42. The cooled perfusate can be cooled via ice and/or a nitrogen gas mixture (e.g., 95% N2 and 5% CO2) (not shown) attached to the second perfusate reservoir 32. The controller 24 can pump cooled perfusate through the second perfusate reservoir 32 through at least one conduit 19 via the second infusion pump 42 in order to adjust-down the temperature of the store of perfusate that will be recirculated back to the graft 14 if indicated based on feedback from one or more sensors as described herein. The third perfusate reservoir 34 can be adapted to hold overflow perfusate from the first perfusate reservoir 30; for example, which can be pumped from the latter if desired to help dilute the concentration of one or more analytes in the perfusate store in the first reservoir 30 from which the graft 14 is to be perfused. The at least one detection device 20 can be within the first perfusate reservoir 20 and/or the venous tube 18b to measure the concentrations of analytes that may accumulate in the perfusate from the implanted biological tissue graft 14 during perfusion. Concentrations of analytes above predetermined concentration thresholds can have negative effects on perfusion on the implanted biological tissue graft 14. The controller 24 can pump the perfusate from the first perfusate reservoir 30 to the third perfusate reservoir 34 via at least one conduit 19 and a third infusion pump 44 in response to detecting a concentration of analytes above a predetermined threshold in the perfusate. Optionally, the controller 24 can pump a portion of the perfusate out of the first perfusate reservoir 30 into the third perfusate reservoir 34 and/or pump cooled perfusate, containing no extra analytes, from the second perfusate reservoir 32 into the first perfusate reservoir 30.

The first, second and third perfusate reservoirs 30, 32, 34 may have any dimension to accommodate a volume of perfusate suitable for use with a portable and patient-wearable system. In a preferred embodiment, the first, second and third perfusate reservoirs 30, 32, 34 may contain up to 1 L of perfusate or fluid. The first, second and third perfusate reservoirs 30, 32, 34 may be removable and replaceable. In a preferred embodiment, at least one of the first, second and third perfusate reservoirs 30, 32, 34 can be replaced daily.

Referring again to FIG. 2, the controller 24 can pump the perfusate from the first perfusate reservoir 30 through at least one conduit 19 via a pump 46 (e.g., a peristaltic pump or roller pump) through an oxygenator 48, shown in greater detail in FIG. 4. The oxygenator may have a flow range of about 1 to about 70 mL/min, about 5 to about 60 mL/min, or about 10 to about 50 mL/min. The oxygenator 48 can be in fluid communication with a heating element 41, an oxygen-based gas mixture 50 (e.g., 97.5% 02 and 2.5% CO2) and humidifier 52. The oxygen-based gas mixture 50 can be humidified by passing through humidifier 52 before entering oxygenator 48. The oxygen-based gas mixture 50 can be stored in, for example, a concentrator or a gas tank. The oxygen-based gas mixture 50 can oxygenate the perfusate as it passes through the oxygenator (via at least one conduit 19) by, for example, the gas mixture combining with an oxygen carrier in the perfusate. The oxygenator 48 can include an outlet for excess gas. Referring to FIG. 4, the oxygenator 48 can also include a fluid inlet and a fluid outlet that can allow a fluid, such as water, to flow from heating element 41 through the oxygenator to facilitate heating and/or cooling the perfusate to a normothermic temperature as the perfusate travels from the first perfusate reservoir 30 to the implanted biological tissue graft 14. A perfusate that can mimic or nearly mimic the oxygen saturation of healthy patient's blood is an important part of maintaining a near physiologic environment for an implanted biological tissue graft because biological tissues require oxygen to survive. A physiologic level of blood oxygen saturation can be between 60% and 100%, 80% and 100%, 90% to 100%, or 95% to 100% depending on if venous oxygen saturation or arterial oxygen saturation is measured. Venous oxygen saturation levels can be lower than arterial oxygen saturation levels without ischemia occurring.

Referring again to FIG. 2, the controller 24 (via pump 46) can move the oxygenated perfusate to the implanted biological tissue graft 14 via the arterial tube 18a. Before the perfusate enters the implanted biological tissue graft 14, the arterial tube 18a can split (e.g., using a flow regulator 47) so a portion of the perfusate in the arterial tube 18a can flow into a sampling manifold 54 and a portion of the perfusate in the at arterial tube 18a can flow into the implanted biological tissue graft 14. The portion of the perfusate that enters the sampling manifold 54 can also be overflow perfusate. The sampling manifold 54 can include an outlet for a user of the system 10 to take a manual sample of the perfusate. The sampling manifold 54 can also be connected to the first perfusate reservoir 30 via at least one conduit 19, to complete the circuit of the perfusate that does not perfuse the implanted biological tissue graft 14 because it was diverted to the sampling manifold 54. The controller 24 can determine the rate at which the perfusate flows to the sampling manifold 54 via the flow regulator 47.

The controller 24 can pump perfusate through arterial tube 18a into the implanted biological tissue graft 14 through a cannula in a major artery of the implanted biological tissue graft 14. The perfusate can then perfuse through the implanted biological tissue graft 14, optionally facilitated by pump 46, and out through a cannula in a major vein of the implanted biological tissue graft 14, into the venous tube 18b, into a negative pressure venous return system 67, back into the venous tube 18b into a perfusate collection receptacle (e.g., a perfusate reservoir). The major vein of the implanted biological tissue graft is in fluid communication with the first perfusate reservoir 30 via the venous tube(s) 18b and the negative pressure venous return system 67, where used perfusate can re-enter the first perfusate reservoir 30, thereby completing the machine perfusion circuit.

The introduction of a venous cannula into the major vein of the biological tissue graft 14 may create resistance in the form of back pressure that could oppose the flow of perfusate and result in vein collapse. Additionally, the length of the venous return tube 18b and changes in orientation of the venous tube 18b as the patient ambulates may also result in pressure differentials affecting venous return. System 10 may include a negative pressure venous return system 67 to compensate for the pressure differentials with negative pressure, improve venous return by preventing vein collapse, and ensure that the flow of used perfusate is unimpeded. The negative pressure venous return system 67 may be provided at the second end of the venous cannula or at any point along the length of the venous tube 18b between the venous cannula and the first perfusate reservoir 30.

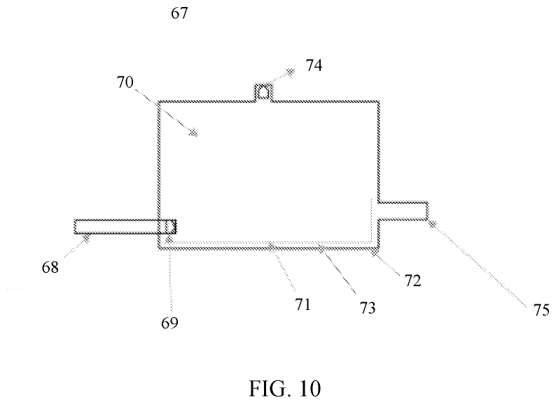

The negative pressure venous return system 67 may regulate the flow rate of the used perfusate, the pressure in the venous return circuit, or a combination thereof. Referring to FIG. 10, the negative pressure venous return system 67 can comprise an enclosure that defines a chamber 70. An inlet 68 to the negative pressure venous return system 67 is positioned in one wall and an outlet 75 to the system is positioned on an opposite wall. Additionally, a pressure-relief valve 74 is fitted in a wall (top wall as illustrated) defining the chamber 70, and is configured to be a normally closed valve that will open at a cracking pressure defining a threshold vacuum within chamber 70, so that the degree of vacuum cannot exceed the threshold. In operation, used perfusate is drawn from the second end of the venous cannula or the venous tubing into an inlet 68 of the negative venous return system 67. Used perfusate passes through the inlet 68 and a one-way valve 69 into chamber 70. The one-way valve 69 is configured to permit used perfusate to enter chamber 70 and to prevent perfusate from flowing backward from the chamber 70 into the inlet 68.

The negative pressure venous return system 67 may be operated with or without a vacuum to control the rate at which the used perfusate is drawn through the system. When the negative pressure venous return system 67 is vacuum-operated, a pump (e.g., a roller pump or a peristaltic pump) may be included in-line downstream of outlet 75 and upstream of the first perfusate reservoir 30 to draw a vacuum against the outlet 75 of the venous return system 67, thereby motivating venous return flow from the graft 14 through the venous return system 67 at a controlled rate. Alternatively, if no vacuum is to be drawn, then the pump 46 configured to deliver perfusate to the graft via the arterial tube 18a may be sufficient regulate the flow of used perfusate through the negative venous return system 67 at a desired rate.

When vacuum is used, the pressure valve 74 will effectively regulate the degree of negative pressure that can be drawn within the chamber 70, and thus effectively against the venous return tube 18b leading from the graft 14, so that the negative pressure drawn thereon cannot exceed a threshold level of vacuum. The threshold level of vacuum at which the pressure valve 74 will crack to admit external air (or other gas) can be selected or tuned to correspond to a degree of vacuum at which the venous return tube 18b will remain reliably patent; i.e. so that it will not be collapsed by the force of vacuum. The one-way pressure valve 74 may also contain a filter (i.e., a HEPA filter) to prevent bacteria or other undesired contaminants in the external environment from entering the closed-loop fluid circuit. The one-way pressure valve 74 may be adjusted manually, such as by a twisting action, or may be adjusted automatically (electrically), in order to regulate its threshold cracking pressure (degree of vacuum). The chamber 70 may contain one or more pressure sensors in operative communication with the controller 24 that are configured to monitor the pressure in the chamber 70 and to adjust the valve 74 (in case it is electrically adjustable) to ensure that the degree of vacuum therein remains within a threshold range. When the one-way pressure valve 74 is automatically controlled, it and pressure sensors in the chamber 70 may operate in a feedback loop with the controller 24 to measure pressure in the chamber 70 and automatically adjust the one-way pressure valve 74 to maintain the threshold pressure value range in the chamber 70.

One or more inner walls 71 are disposed within the chamber 70 and are offset from a wall 72 of the negative pressure venous return system 67, such that a narrow channel 73 is formed therebetween. When more than one inner wall 71 is employed, the additional inner wall(s) may be offset from one another in chamber 70 to create a series of narrow channels 73 communicating between a sink at the base of the chamber where perfusate will pool, and the outlet 75 through which the used perfusate will leave the chamber 70 and enter the downstream venous tubing to be returned to the first perfusate reservoir 30. In this manner, capillary action helps to ensure that substantially only the liquid perfusate solution pooled at the bottom of the chamber 70 will be drawn out through the outlet 75, and not air or other gas within the headspace of that chamber, which may diminish the efficiency of the system.

Specifically, referring to FIG. 10 (which illustrates the negative pressure venous return system 67 positioned horizontally), when used perfusate enters the chamber 70 through inlet 68, at least a portion of the used perfusate flows from chamber 70 into the narrow channel 73. The used perfusate is drawn through the narrow channel 73 and passes through the outlet 75. This configuration creates a capillary effect ensuring that used perfusate continues to be drawn out of the system (through the pump controlling the flow rate, the regulated pressure or both) irrespective of the orientation of the negative pressure venous return system 67 as the patient ambulates. For example, when the negative pressure venous return system 67 is vertically oriented, at least a portion of the used perfusate entering the chamber 70 will flow from the chamber into the narrow channel 73. Through the capillary effect, the used perfusate will be drawn (by the pump at the controlled flow rate, the regulated pressure, or both) through the narrow channel 73 and out through the outlet 75 as the chamber fills with perfusate.

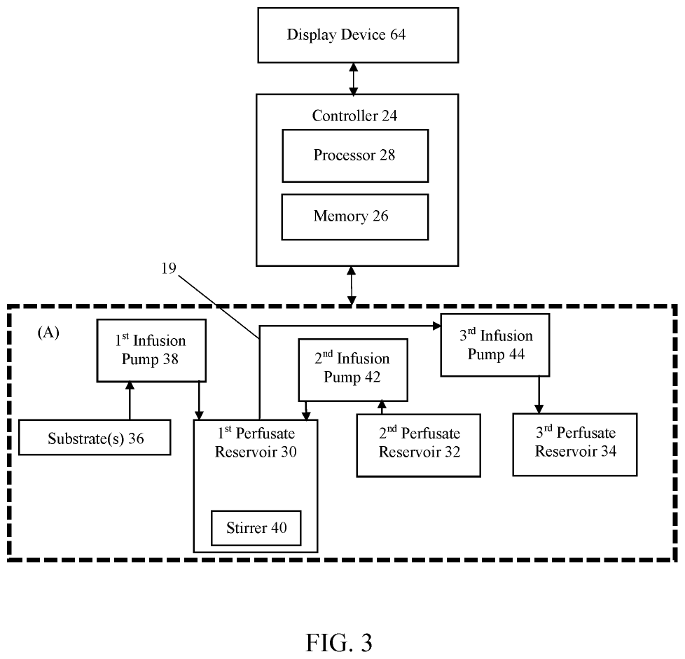

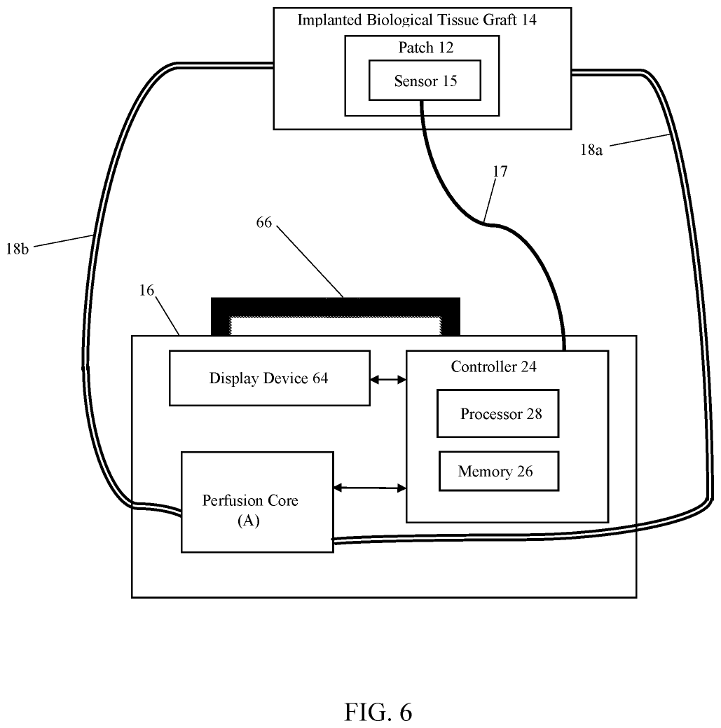

Returning to FIGS. 2-3, the system 10 also includes a display device 64 connected to the controller 24. The display device 64 may be included within the perfusion assembly 16 as shown in FIG. 3, or it may be physically positioned outside of the perfusion assembly 16 but operatively connected and/or attached thereto. The display device 24 can be adapted to display measurements from the one or more detection devices 20 positioned throughout the system 10 and discussed above. The display device 64 can also be adapted to display alerts when a parameter detected by the at least one sensor 15 remains within a predetermined first threshold range for that parameter despite cessation of perfusion to the implanted biological tissue graft 14, or if a parameter detected by the one or more detection devices 20 is outside of the at least one predetermined threshold for that parameter. The alert can be at least one of visual, auditory, or tactile. As illustrated in FIG. 6, the display device 64 can also contain a user interface with operational controls so that a user of the system 10 may input values, for example, the parameter thresholds, predetermined durations, time intervals, and the type, size or weight of the implanted biological tissue graft 14, into the controller 24.

The perfusion assembly 16 is patient-wearable and human-portable, such that the components thereof are confined within dimensions such that they may be worn and/or carried by the patient. For example, the perfusion assembly may be confined within a structure, such as a box, a capsule or other container that is human-portable to be worn and/or carried by the patient. The perfusion assembly 16 may be placed into a backpack, a messenger bag, or other carrying case to be human portable, e.g. having a shoulder strap so that it can be easily carried or worn by an ambulatory patient. For example, FIG. 6 shows an exemplary view of system 10, wherein the biological tissue graft 14 is connected to the perfusion assembly 16, which is housed within a carrying case or bag having a strap 66, or one or more belts and/or handles, to facilitate the patient-wearable and human-portable features of the system 10. FIG. 6 is only one example configuration of the system 10, and a person of ordinary skill in the art would understand that system 10 can have many other configurations that are human-portable and patient-wearable. The system may also include a portable power source adapted to power the system 10 without need for the system 10 to stay permanently attached to a stationary power source (e.g., a wall or floor outlet).

Wearable portability of the system 10 in combination with longer preservation times from normothermic machine perfusion can reduce surgical transplantation time and allow an implanted biological tissue graft 14 to naturally vascularize to and with the patient's body as the patient goes about his or her everyday life without the need to remain hospitalized under continuous medical supervision. The system 10 described above permits an implanted biological tissue graft 14 to be preserved and sustained for at least four weeks (one month) or until the body neovascularizes the reattached tissue.

### III. Methods

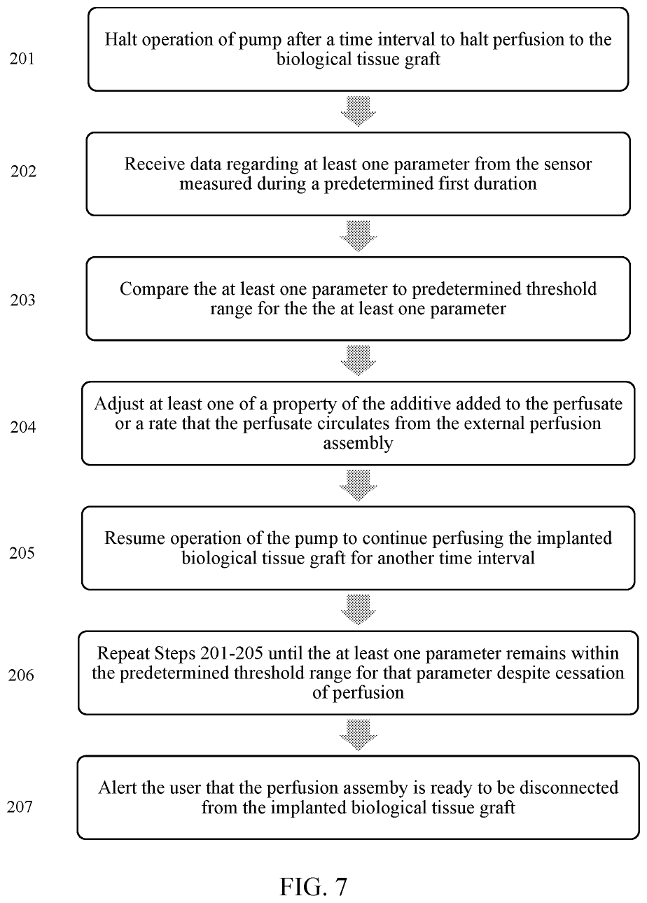

Another aspect of the present disclosure can include a method (FIG. 7) for preserving an implanted biological tissue graft with a portable, patient-wearable system 10, or any of its aspects described above. The method is illustrated as process flow diagrams with flowchart illustrations. For purposes of simplicity, the method is shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the method. The method can be implemented by at least a portion of the system shown in any one of FIGS. 1-6.

FIG. 7 illustrates a method for preserving an implanted biological tissue graft in vivo. Wearable portability of the perfusion system used to execute the method of FIG. 7 in combination with longer preservation times from normothermic machine perfusion can allow an implanted biological tissue graft (e.g., a skin tissue graft) to naturally vascularize with the patient's body without the need to remain hospitalized under continuous medical supervision so that the patient can go about his or her everyday life as normal.

As a preliminary step, the perfusion assembly, which is external to a patient's body, is connected to a detached biological tissue graft harvested from a donor or the patient by cannulating arterial and venous tubes of the perfusion assembly to a major artery and a major vein, respectively, of the graft via respective cannula. Once the perfusion assembly has been connected to the detached biological tissue graft, the perimeter of the detached biological tissue graft is sutured to a defect at an external surface of the patient's body to form an implanted biological tissue graft. A patch including at least one sensor is applied to the external (outward-facing) surface of the implanted biological tissue graft, such that the at least one sensor is in direct contact with the external surface of the graft. A user (i.e., a patient or a medical caregiver) activates the system, for example via operational controls on the display device, to initiate the flow of perfusion through the perfusion assembly to the implanted biological tissue graft via the arterial tube. Perfusate flow to the major artery of the biological tissue graft is pressure controlled. In one embodiment, the system may be initiated by slowly increasing the flow of perfusate to the graft to reach a minimum pressure of 40 mmHg. Because vasoconstriction occurs in the graft vessels immediately after cannulation, the flow of perfusate may be initiated at a rate of 3 mL/min and maintained for between 10 minutes to 15 minutes to stabilize the pressure. Thereafter, the flow rate may be adjusted from between 2 mL/min to 30 mL/min every 10 minutes to 15 minutes until the minimum pressure is achieved and maintained. Spent perfusate is collected from the implanted biological tissue graft via the venous tube and transported to the perfusion assembly. In this manner, the arterial and venous tubes yield a closed fluid circuit between the perfusion assembly and the implanted biological tissue graft. Once perfusion has been initiated, a controller including a processor executes an algorithm stored in memory to perform the following steps.

At Step 201, the controller halts the operation of at least one pump at a first time interval for a predetermined first duration of time. The pump may be, for example, an infusion pump for controlling the amount of an additive added to a perfusate reservoir or the amount of perfusate added to or removed from a perfusate reservoir, or a pump responsible for controlling the rate at which perfusion flows from the perfusion assembly to the implanted biological tissue graft. In any event, halting the operation of any pump in the system stops the provision of perfusate to the implanted biological tissue graft. The first time interval can be programmed into the controller's memory. For example, the controller's memory may halt the pump after the expiration of 1 hour, 12 hours, 24 hours, 36 hours, 48 hours, 60 hours, 72 hours, 84 hours, or so on. Alternatively, the time interval can be input by a user before perfusion has been initiated or during perfusion. For example, the controller may signal the display device to prompt the user (i.e., the patient or a medical caregiver) to input a time interval several hours after perfusion has begun. In one embodiment, a first time interval of 72 hours is used to create a period of gradient to force the body to neovascularize. The predetermined first duration of time may also be programmed into the controller's memory, or may be input by a user via the display device in the same manner as the time interval. In one embodiment, the predetermined first duration of time is 15 minutes.

At Step 202, the controller receives data regarding a first parameter measured by at least one sensor in direct contact with the external surface of the implanted biological tissue graft during the predetermined first duration. The first parameter is a physiologic parameter (e.g., temperature, pressure, oxygen saturation, etc.) and the data may include a single value (i.e., a single temperature measurement), or two or more values measured at time points within the predetermined first duration. In one embodiment, the sensor continuously measures the first parameter, and the controller only begins receiving data regarding said first parameter from the sensor once the pump has halted. In another embodiment, the controller signals the sensor to begin measuring the first parameter for the predetermined period of time once the pump has halted, to communicate the data regarding the first parameter to the controller, and to cease measuring the first parameter after the predetermined period of time. In an embodiment where the system includes a sensor array, the controller processes the respective values of the first parameter from each sensor in the array (i.e., by calculating the average, mean or median value).

At Step 203 the controller compares the data regarding the first parameter received from the sensor to a predetermined first threshold range for the at least one parameter. The predetermined first threshold range for the first parameter is programmed into memory and represents a parameter range for vascularized biological tissue. For example, if the first parameter is oxygen saturation, the predetermined first threshold range for oxygen saturation may be set at 80% oxygen saturation. In one embodiment, the controller may compare data for a single first parameter (i.e., temperature, oxygen saturation, and/or color) to a predetermined threshold range for that parameter. The data regarding the first parameter may include a single value (i.e., one temperature reading) or multiple values measured at distinct time periods or continuously during the predetermined period of time. When the data includes multiple values, the controller may, for example, calculate the average, mean or median of said values for comparison with a predetermined threshold range for the at least one parameter. In another embodiment, the controller may compare data for one or more parameters (i.e., temperature, oxygen saturation and/or color) measured by one or more sensors with predetermined threshold ranges for the respective parameters.

At Step 204, based on the comparison in Step 203, the controller adjusts at least one of: (i) a property of the perfusate, or (ii) a rate that the perfusate circulates from the perfusion assembly to the biological tissue graft. The property of the perfusate may be a concentration of an additive in the perfusate supplied from a separate additive reservoir of the external perfusion assembly, and said adjustment may include increasing, decreasing or maintaining the concentration of an additive added to the perfusate and/or the rate at which the perfusate circulates. As the patient's body naturally vascularizes the implanted biological tissue graft, the implanted biological tissue graft's dependence on the perfusate for sustenance decreases. Thus, if the data regarding the at least one parameter measured by the sensor during the predetermined first duration is within the predetermined range for that parameter, then the controller determines that the implanted biological tissue graft is being naturally vascularized by the patient's body and is less dependent (or, in some cases, no longer dependent) on perfusate for sustenance. Accordingly, the controller may adjust a property of the perfusate (i.e., the concentration of additive added to the perfusate supplied from a separate additive reservoir of the external perfusion assembly) and/or the rate at which perfusate is circulated to the implanted biological tissue graft down. Conversely, if the data for the at least one parameter obtained by the sensor after the pump has been halted is outside of the predetermined range for that parameter, then the controller determines that the implanted biological tissue graft has not yet been adequately vascularized by the body and remains dependent on perfusate for sustenance. In that instance, the controller may adjust the property of the perfusate (i.e., the concentration of additive added to the perfusate supplied from a separate additive reservoir) and/or the rate at which the perfusate is provided to the implanted biological tissue up, or maintain them at their existing settings. Taking the oxygen saturation example above, if the controller determines during the comparison step 203 that the data regarding oxygen saturation received from the sensor is greater than the first threshold range of 80%, then the controller will reduce the rate of perfusion by 3 mL/min to create a gradient for neovascularization.

At Step 205, the controller resumes operation of the at least one pump to resume circulation of perfusate to the implanted biological tissue graft for another time interval. Each subsequent time interval may be the same as the first time interval, or they may be different from the first time interval. In one embodiment, all time intervals may be 1 day, 2 days, or 3 days. In another embodiment, the first time interval may be 1 day, a second time interval may be 1 day, and a third time interval may be 3 days.

At Step 206, steps 201-205 are repeated. Each subsequent predetermined duration may be the same as the predetermined first duration, or they may be different. In one embodiment, all predetermined durations are 15 minutes. In another embodiment, the predetermined first duration of time may be 1 minute, a predetermined second duration of time may be 2 minutes, a predetermined third duration of time may be 3 minutes, and so on. Steps 201-205 are repeated until the first parameter remains within the predetermined first threshold range despite cessation perfusion of the biological tissue graft for a predetermined final duration. The predetermined final duration may be set by a user or may be programmed into the controller's memory as detailed above. When programmed into memory, the predetermined final duration may be specific to a type, size or weight of an implanted biological tissue graft.

At Step 207, when the controller determines that the data regarding the first parameter at the predetermined final duration of cessation of perfusion of the implanted biological tissue graft remains within the predetermined first threshold range, the controller sends an alert (i.e., via the display device) to the patient and/or the medical caregiver indicating that the perfusion assembly is ready to be disconnected from the implanted biological tissue graft. At this step, the controller has determined that the implanted biological tissue graft has been sufficiently vascularized by the patient's body, such that the implanted biological tissue graft no longer requires sustenance from the perfusate.

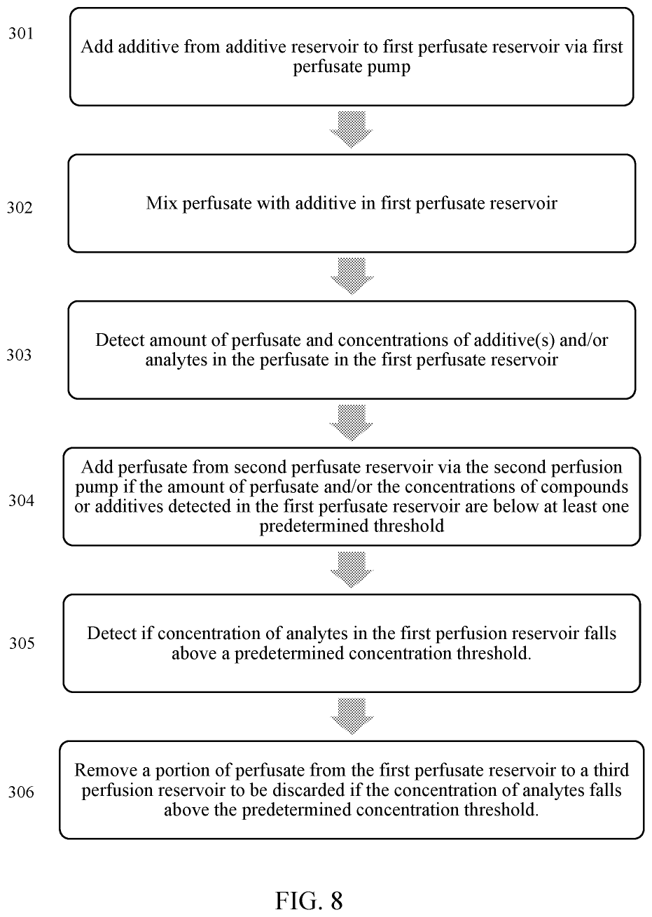

FIG. 8 illustrates a method for exchanging perfusate within a portable, patient-wearable perfusion system having a perfusion core that is being used to preserve and sustain an implanted biological tissue graft. The perfusion core can include a first, second, and third perfusate reservoir each adapted for holding perfusate. The perfusion core can also include a first, second, and third infusion pump. The first, second, and third infusion pumps can each be connected to the controller. At Step 301, the controller can pump, by the first infusion pump, one or more additives, such as a substrate, nutrient, electrolyte (e.g., Na+, K+, Ca++, etc.), or other compound (e.g., sodium bi-carbonate NaHCO₃), from one or more additive reservoirs, into the first perfusate reservoir. At Step 302, the controller can mix, by a stirrer such as a magnetic stirrer, the perfusate in the first perfusate reservoir with the additive(s) added to the first perfusate reservoir. The stirrer can be connected to the controller and located in and/or adjacent to the first perfusate reservoir. At Step 303, the controller can detect, by the at least one detection device, when the amount of perfusate and/or concentrations of compounds (i.e., analytes) and/or additive(s) in the perfusate are outside of at least one predetermined amount or at least one predetermined concentration level (e.g., outside at least one predetermined threshold).

At Step 304, the controller can add, by the second infusion pump, perfusate from the second perfusate reservoir to the first perfusate reservoir when the amount of perfusate and/or concentrations of compounds (i.e., analytes) or additive(s) detected in the first perfusate reservoir are below the at least one predetermined threshold. The second perfusate reservoir can hold cooled perfusate (e.g., cooled on ice and/or with a nitrogen gas mixture) with no additional compounds added. The addition of the cooled perfusate to the first perfusate reservoir can increase the concentrations of perfusate compared to the substrates and analytes detected by the at least one detection device without having to remove perfusate from the first perfusate reservoir.

At Step 305, the controller can detect, by the at least one detection device, that a concentration of analytes in the perfusate is above a predetermined concentration threshold after the perfusate has perfused the implanted biological tissue graft. Analytes enter the perfusate from the implanted biological tissue graft and can be detrimental to effective perfusion of the implanted biological tissue graft when their concentration in the perfusate is above a predetermined level. At Step 306, the controller can remove, via the third infusion pump, at least a portion of the perfusate comprising the analytes from the first perfusate reservoir to the third perfusate reservoir (e.g., to be discarded or cleaned of analytes by a user). The controller can then add clean, cooled perfusate, via the second infusion pump, and additional substrates, via the first infusion pump to the first perfusate reservoir to maintain an amount of perfusate circulating through the system. The controller may detect, by at least one detection device, when the third reservoir is filled with perfusate and may alert the user that the third reservoir is ready to be replaced or emptied. Similarly, the controller may detect, by at least one detection device, when the second perfusate reservoir is empty, and may alert the user to replace or refill the second perfusate reservoir.

The controller may repeat steps 301-306 at predetermined time intervals, for example every 12 hours. Alternatively, the controller may continuously repeat steps 301-306.

From the above description, those skilled in the art will perceive improvements, changes and modifications. Such improvements, changes and modifications are within the skill of one in the art and are intended to be covered by the appended claims.

1. A portable, patient-wearable perfusion system comprising:
a patch configured to cover at least a portion of a biological tissue graft implanted within a defect in a patient, the patch comprising a first sensor adapted to detect a first parameter relating to a physiological state of the biological tissue graft in vivo;
a human-portable perfusion assembly configured to be worn or carried by the patient, the perfusion assembly comprising:
a reservoir for a perfusate;
a pump; and
a controller operatively coupled to the sensor and configured to operate the pump to control said physiologic state of the tissue graft based on data regarding the first parameter received from the sensor;
an arterial cannula configured to cannulate a major artery of the biological tissue graft to facilitate delivery of perfusate to the graft from the perfusion assembly; and
a venous cannula configured to cannulate a major vein of the biological tissue graft to facilitate return of spent perfusate from the graft back to the perfusion assembly;
wherein the arterial cannula and the venous cannula are configured to yield a closed fluid circuit for the perfusate to circulate through the biological tissue graft.

2. The system of claim 1, further comprising:
a suction pump disposed in-line between the venous cannula and the perfusion assembly and configured to draw the perfusate from the graft via the venous cannula, and
an enclosure defining a chamber disposed in-line between the venous cannula and the suction pump, the enclosure configured to ensure that a degree of suction drawn against the venous cannula by the suction pump cannot exceed a predetermined threshold.

3. The system of claim 1, wherein the first sensor comprises:
a tissue oximeter configured to measure oxygen saturation in the biological tissue graft;
a temperature sensor configured to measure a temperature of the biological tissue graft; or
an optical color sensor configured to detect a color of the biological tissue graft.

4. The system of claim 1, said patch comprising a first sensor array that comprises a plurality of said first sensors distributed across a surface of the patch, which are thus adapted to detect respective values of said first parameter from a multitude of corresponding locations in the biological tissue graft, the controller being configured to process the respective values of said first parameter and to control said physiologic state of the tissue graft based thereon.

5. The system according to claim 4, said patch further comprising a second sensor array that comprises a plurality of second sensors distributed across said surface and adapted to detect respective values of a second parameter relating to the physiologic state of the biological tissue graft from a multitude of corresponding locations therein, the controller being configured to process the respective values of the second parameter, together with those of the first parameter, and to control said physiologic state of the tissue graft based thereon.

6. The system according to claim 5, said first sensors being temperature sensors, said first parameter being temperature, said second sensors being tissue oximeters, said second parameter being oxygen saturation.

7. The system of claim 1, wherein the perfusion assembly further comprises a source of a gas mixture connected to the oxygenator and configured to provide a quantity of the gas mixture to the perfusate and to maintain a desired pH in the perfusate.

8. The system of claim 1, wherein the venous cannula comprises an insertion end comprising an attachment feature configured to secure the insertion end to an interior lumen of the major vein of the biological tissue graft by friction or press-fit.

9. The system of claim 2, said enclosure comprising an interior wall that at least partially defines a capillary passageway to facilitate flow of perfusate via capillary action to an outlet of the chamber.

10. The system of claim 9, said enclosure further comprises a valve configured to admit air or other gas into said chamber in order to ensure said predetermined threshold of suction therein.

11. The system of claim 9, further comprising a one-way valve disposed at an inlet of said enclosure and configured to ensure that perfusate cannot flow backward from the enclosure toward the venous cannula.

12. A method of preserving an implanted biological tissue graft, comprising:
connecting an external perfusion assembly located outside of a patient's body to a major artery and a major vein of a biological tissue graft via respective arterial and venous cannulas, thereby establishing a closed fluid circuit for a flow of perfusate between the tissue graft and the perfusion assembly; and
perfusing the biological tissue graft in vivo via perfusate circulating through said closed fluid circuit.

13. The method of claim 12, said external perfusion assembly comprising:
a reservoir for said perfusate;
a pump; and
a controller operatively coupled to a sensor in a patch that covers at least a portion of the implanted biological tissue graft;
wherein the sensor detects a first parameter concerning a physiologic state of the in vivo biological tissue graft, and the controller operates the pump to control the physiologic state thereof based on data from the sensor regarding the first parameter.

14. The method of claim 13, said external perfusion assembly further comprising an additive reservoir for an additive to be added to said perfusate.

15. The method of claim 13, wherein the controller executes an algorithm to carry out the following steps:
(a) after a time interval, halt operation of the pump to thereby halt perfusion of the biological tissue graft for at least a predetermined first duration;
(b) receive the data regarding the first parameter from the sensor during the predetermined first duration, and compare that data to a predetermined first threshold range for the first parameter;
(c) adjust at least one of:
i. a property of the perfusate, or
ii. a rate that the perfusate circulates;
(d) resume operation of the pump to resume perfusion of the biological tissue graft; and
(e) repeat steps (a)-(d) until the first parameter remains within the predetermined first threshold range despite cessation of perfusion of the biological tissue graft.

16. The method of claim 15, the property of the perfusate being a concentration of an additive in the perfusate supplied from a separate additive reservoir of the external perfusion assembly.

17. The method of claim 15, wherein the algorithm further configures the controller to:
(f) alert the patient and/or a medical caregiver once the first parameter remains within the predetermined first threshold range despite cessation of perfusion of the biological tissue graft.

18. The method of claim 15, said first parameter being one of the following: temperature of the biological tissue graft, oxygen saturation in the biological tissue graft, or color of the biological tissue graft.

19. The method of claim 12, said external perfusion assembly being human-portable and comprising a portable power source.
